(19) United States
(12) Patent Application Publication
(10) Pub. No.: US 2024/0304310 A1
(43) Pub. Date: Sep. 12, 2024

(54) Diagnostic image translation using a deep neural network with both imaging parameters and diagnostic images as input

(71) Applicant: The Board of Trustees of the Leland Stanford Junior University Office of the General Counsel, Stanford, CA (US)

(72) Inventors: Shreyas S. Vasanawala, Stanford, CA (US); Yan Wu, Mountain View, CA (US)

(21) Appl. No.: 18/597,385

(22) Filed: Mar. 6, 2024

Related U.S. Application Data
(60) Provisional application No. 63/450,225, filed on Mar. 6, 2023.

(51) Int. Cl. G16H 30/40; G06V 10/82; G16H 30/20; G16H 50/20

(52) U.S. Cl. CPC G16H 30/40; G06V 10/82; G16H 30/20; G16H 50/20

(57) ABSTRACT

A deep learning method generates translated images from both diagnostic images acquired with predetermined image acquisition parameters and from the predetermined image acquisition parameters. The translated diagnostic images are generated by applying both the predetermined image acquisition parameters and the diagnostic images as input to the deep neural network in the form of parameter image maps with imaging parameter values at each pixel of the parameter image maps.

### CROSS-REFERENCE TO RELATED APPLICATIONS

This application claims priority from U.S. Provisional Patent Application No. 63/450,225 filed Mar. 6, 2023, which is incorporated herein by reference.

### STATEMENT OF FEDERALLY SPONSORED RESEARCH

This invention was made with Government support under contract DK117354, EB009690, EB026136 awarded by the National Institutes of Health. The Government has certain rights in the invention.

### FIELD OF THE INVENTION

The present invention relates generally to medical diagnostic imaging. More specifically, it relates to techniques for image-to-image translation of diagnostic images using deep learning.

### BACKGROUND OF THE INVENTION

In recent years, deep learning has begun to play an important role in image-to-image translation tasks. In radiological imaging, deep learning has made significant contributions to a variety of applications, including but not limited to quantitative MR parametric mapping and water-fat separation. A common deep learning-based imaging model is typically trained to learn the physical model only from input radiological images without consideration to the values of imaging parameters.

### SUMMARY OF THE INVENTION

We describe here a technique that incorporates the values of imaging parameters as additional input to a deep learning-based radiological imaging model. Thus, not only are radiological images used as input to the deep neural network, but also image maps of the values of critical imaging parameters at every pixel are incorporated into the input to the deep neural network. The inventors have discovered and demonstrated that explicit incorporation of such a priori knowledge as additional input into the network improves prediction accuracy, particularly when flexible imaging parameter values are adopted for data acquisition. Previously, the values of imaging parameters have been used for loss calculation in self-supervised learning, but never as input to the network used for image translation.

Thus, in one aspect, the invention provides a method for diagnostic imaging comprising: performing a diagnostic imaging scan using predetermined image acquisition parameters prescribed in an imaging protocol to produce diagnostic images; and generating translated diagnostic images from the predetermined image acquisition parameters and from the diagnostic images using a deep neural network. The translated diagnostic images are generated by applying both the predetermined image acquisition parameters and the diagnostic images as input to an input layer of the deep neural network. The predetermined image acquisition parameters are input to the deep neural network in the form of parameter image maps with imaging parameter values at each pixel of the parameter image maps. The translated diagnostic images are produced as output from an output layer of the deep neural network.

In one embodiment, the diagnostic imaging is magnetic resonance imaging, the diagnostic images are T₁-weighted images acquired using variable flip angles with or without B₁ map, the predetermined image acquisition parameters comprise variable flip angles, and the translated diagnostic images comprise a T₁ map. The T₁-weighted images may be acquired with distinct flip angles. The translated diagnostic images may comprise an uncompensated T₁ map. In this case, the nominal variable flip angles are additional input into the neural network. Alternatively, the translated diagnostic images comprise a compensated T₁ map that takes into account B₁ inhomogeneity. In this case, the predetermined image acquisition parameters (i.e., nominal flip angle) are combined with a B₁ map to produce actual variable flip angles that are input into the neural network in the form of a nominal flip angle modulated by the B₁ map. The translated diagnostic images may also comprise a ρ map.

In other embodiments, the diagnostic imaging is chemical shift encoded magnetic resonance imaging (MRI) using dual echo image acquisition; the diagnostic images are in-phase and out-of-phase complex MRI images, the predetermined image acquisition parameters are echo times, and the translated diagnostic images are water and fat images.

In yet other embodiments, the diagnostic images may be modified look-locker imaging based T₁ weighted images, multi-echo T₂ or T_{2*} weighted images, continuous wave T_{1ρ} weighted images, adiabatic T_{1ρ} weighted images. The predetermined image acquisition parameters may be inversion times, echo times, spin-lock times, or number of adiabatic inversion recovery pulses. The translated diagnostic images may comprise T₁ map, T₂ or R₂ map, T_{2*} or R_{2*} map, and T_{1ρ} map.

The deep neural network may be a convolutional network, attention convolutional network, pure attention network, or generative adversarial network.

In some embodiments, the deep neural network may be trained using training diagnostic images and corresponding translated images generated using a conventional MR image processing technique such as least square fitting for generating quantitative parametric maps, and projected power approach for generating water and fat images.

In other embodiments, the deep neural network is trained using training diagnostic images via self-supervised learning technique comprising inputting the training diagnostic images to the deep neural network to produce estimated translated images as output, generating from the estimated translated images synthetic images using a model-based calculation, and computing a loss function by comparing the synthetic images to the training diagnostic images. In this way, reference translated images are no longer needed.

### BRIEF DESCRIPTION OF THE DRAWINGS

FIG. 1 is a schematic diagram illustrating a method for diagnostic imaging translation according to an embodiment of the present invention.

FIG. 2A is a schematic diagram that compares three methods for image translation in the case of uncompensated T₁ mapping that generates a T₁ map from variable flip angle (VFA) images.

FIG. 2B is a schematic diagram that compares three methods for image translation in the case of compensated T₁ mapping that takes B₁ inhomogeneity into consideration, where the nominal flip angles (FAs) are combined with B₁ map and used as part of the network input.

FIG. 2C is a schematic diagram that compares three methods for image translation in the case of dual-echo water-fat separation, where echo times used to acquire the in-phase and out-of-phase images are included as additional network input.

FIGS. 3A-3B are schematic diagrams illustrating self-supervised learning in the case of uncompensated T₁ mapping and compensated T₁ mapping, respectively.

FIG. 4A is a schematic diagram illustrating the architecture of a self-attention convolution network employed for self-supervised T₁ mapping according to an embodiment of the invention.

FIG. 4B is a schematic diagram illustrating the architecture of a single convolutional block in the network of FIG. 4A.

Results demonstrating the performance of embodiments of the present invention are shown in FIGS. 5, 6, 7.

FIG. 5 is a collection of images comparing results of different methods for image translation of T₁ weighted images acquired with flip angles 5°, 10°, 20°, and 30° to uncompensated T₁ maps.

FIG. 6 is a collection of images comparing results of different methods for image translation of T₁ weighted images acquired with flip angles 5°, 10°, 20°, and 30° and a B₁ map to compensated T₁ maps.

FIG. 7 is a collection of images comparing results of different methods for image translation of acquired in-phase images and out-of-phase images to water and fat images.

### DETAILED DESCRIPTION OF THE INVENTION

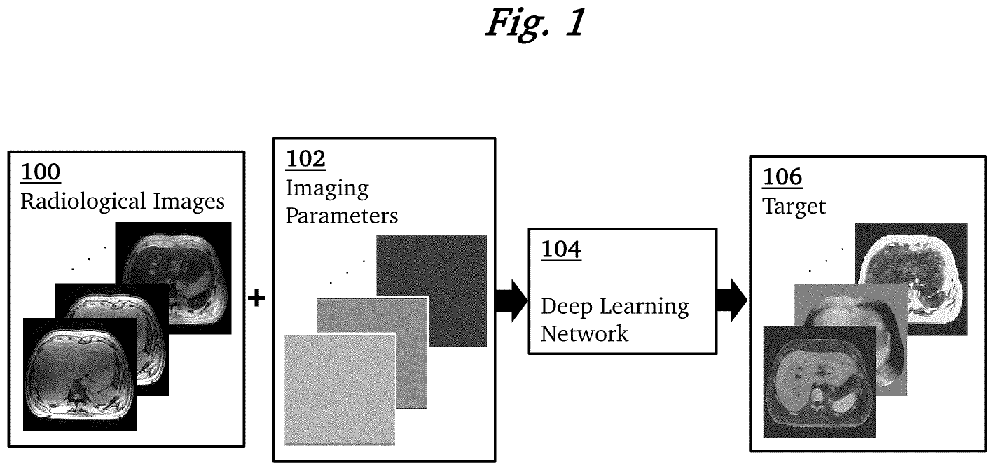

A schematic diagram illustrating a method for diagnostic imaging according to an embodiment of the present invention is shown in FIG. 1. A diagnostic imaging scan is performed to produce diagnostic images 100. The scan is performed using predetermined image acquisition parameters 102 that have been prescribed in an imaging protocol for the scan. In one illustrative example, the diagnostic imaging may be magnetic resonance imaging (MRI), the diagnostic images may be T₁-weighted images acquired with variable flip angles, and the predetermined image acquisition parameters may be flip angles. The techniques of the present invention, however, are not limited to this example, but may include other types of MRI acquisition parameters as well as other types of diagnostic imaging such as computed tomography or ultrasound.

The diagnostic images 100 and corresponding imaging parameters 102 are both applied to an input layer of a deep neural network 104. The predetermined image acquisition parameters 102 are input to the deep neural network in the form of parameter image maps with imaging parameter values at each pixel of the parameter image maps. For example, the predetermined image acquisition parameters may be values of variable flip angles at each pixel. More generally, MRI image acquisition parameters are variables in pulse sequences that determine how radiofrequency (RF) pulses are applied so as to achieve certain image contrast, signal-to-noise ratio, acquisition time, and/or resolution in corresponding MR images. Examples of MRI imaging parameters include echo time (TE), repetition time (TR), inversion time, flip angle, echo train length.

The deep neural network 104 generates as output at an output layer translated diagnostic images 106 from the predetermined image acquisition parameters 102 and from the diagnostic images 100 that were input to the deep neural network. For example, the translated diagnostic images output from the network 104 may be a T₁ map generated from T₁-weighted images and flip angle acquisition parameters input to the network. More generally, the network 104 is trained to perform image-to-image translation, which is the process of computationally transforming images acquired using given image acquisition parameters to image(s) that would have been derived using conventional processing technique (e.g., least square fitting, projected power approach). In the present invention, however, the deep learning-based image-to-image translation is performed by supplementing the input images with imaging parameters.

There are different ways to integrate imaging parameters as network input. As an example, the translated diagnostic images 106 output from the network 104 may comprise an uncompensated T₁ map or a compensated T₁ map that takes into account B₁ inhomogeneity. In the former case, the predetermined image acquisition parameters, i.e., nominal flip angles, 102 are directly included as input. In the latter case, the image acquisition parameters of flip angles are combined with B₁ map to produce actual variable flip angles 102, and actual variable flip angles are input into the neural network 104 in the form of a nominal flip angle modulated by the B₁ map.

This technique can be applied in various radiological imaging modalities, such as MRI, CT, or ultrasound. As an illustrative example, we describe this technique in the context of MRI for quantitative T₁ mapping and water-fat separation. We also compare the technique with existing methods that do not use imaging parameters as supplemental input to a deep neural network.

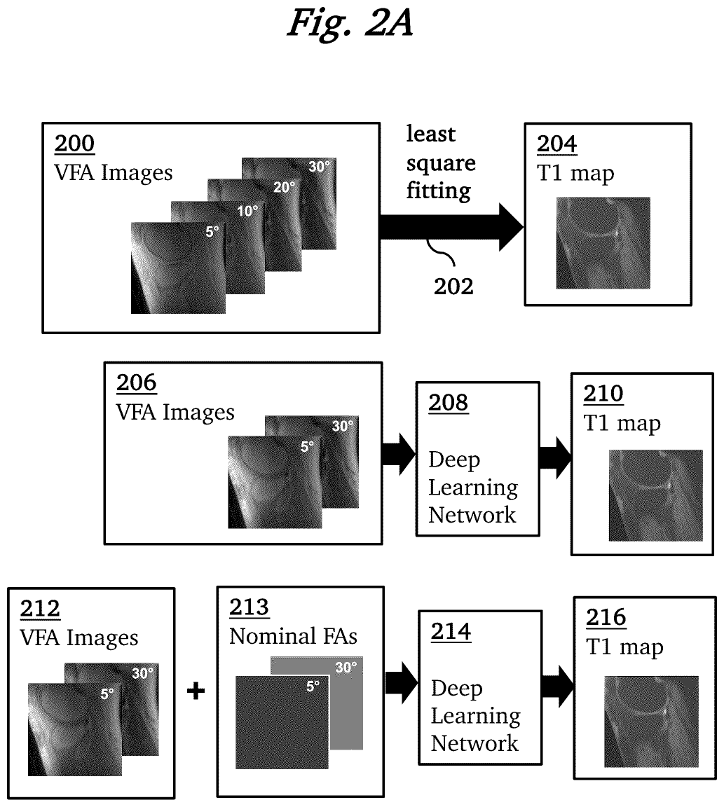

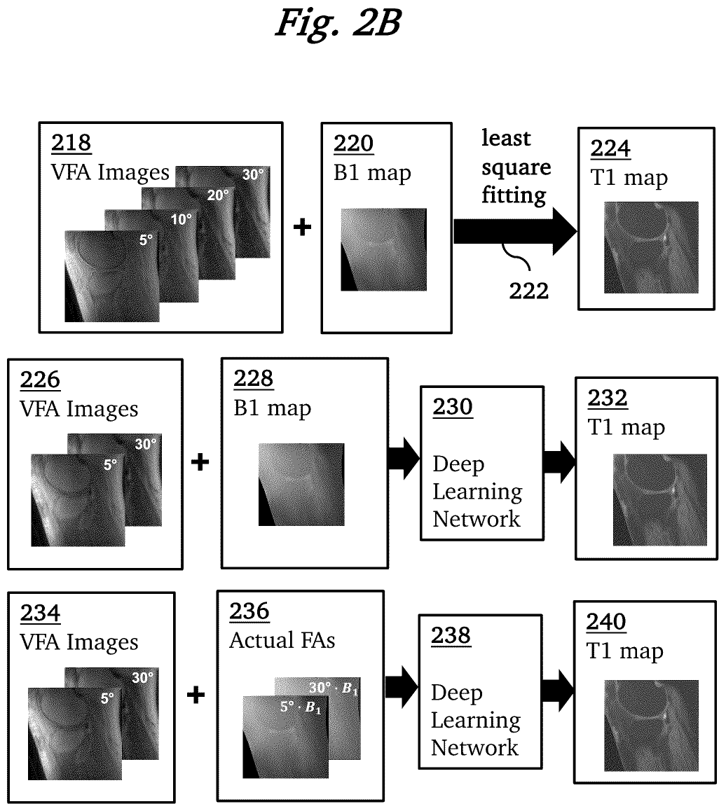

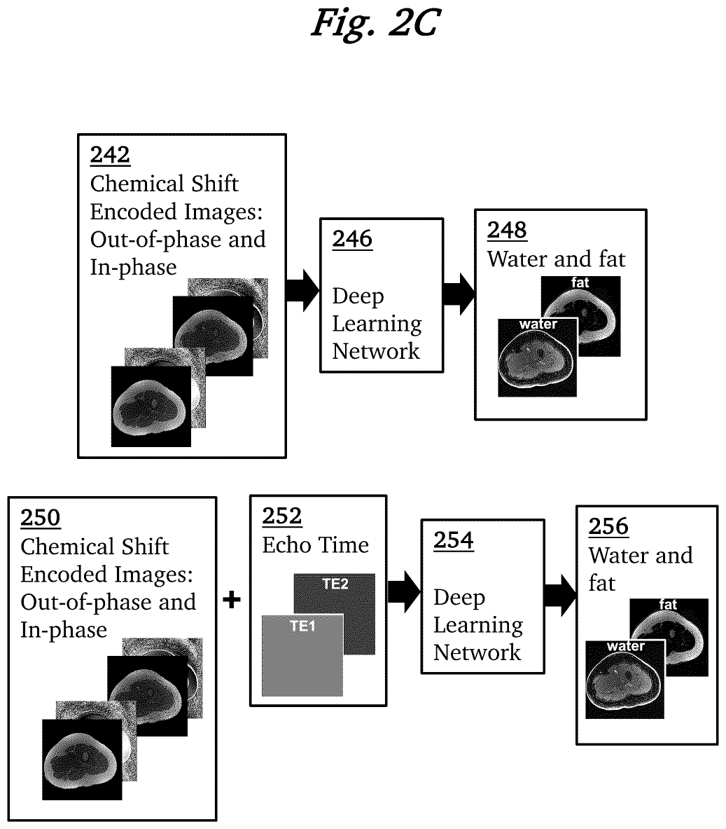

FIGS. 2A, 2B, 2C illustrate three examples of a method that incorporates the values of imaging parameters as additional network input, comparing each to existing methods. FIG. 2A illustrates the case of uncompensated T₁ mapping that generates a T₁ map from a reduced number of variable flip angle (VFA) images, where the nominal flip angles prescribed in the imaging protocol are included as additional network input. FIG. 2B illustrates the case of compensated T₁ mapping that takes B₁ inhomogeneity into consideration, where the nominal flip angles (FAs) are combined with B₁ map and used as part of the network input. FIG. 2C illustrates the case of dual-echo water-fat separation, where echo times used to acquire the in-phase and out-of-phase images are included as additional network input.

As shown in FIG. 2A, four variable flip angle (VFA) images 200 are acquired using an ultrashort echo time (UTE) cones sequence with flip angles of 5°, 10°, 20°, and 30° respectively, echo time of 32 μs, and time of repetition of 20 ms. Using conventional least squares fitting techniques 202, an uncompensated ground truth T₁ map 204 is extracted from the four VFA images based on

$$S = ρ \cdot sin ( α ) \frac{1 - {e}^{- TR / {T}_{1}}}{1 - cos ( α ) \cdot {e}^{- TR / {T}_{1}}} ,$$

and smoothed via a 3D Gaussian kernel.

Also shown in FIG. 2A are deep learning-based T₁ mapping models. The baseline model is a deep neural network 208 that is trained to predict an uncompensated T₁ map 210 from only two VFA images 206 (e.g., acquired with flip angles of 5° and 30°). In contrast with the baseline model, the present deep learning model 214 predicts the translated images 216 from the same two VFA images 212 supplemented with additional imaging parameter maps 213 that provide the nominal flip angles (5° and 30°) at every pixel. In our experimental tests, both networks 208 and 214 have the same architecture (e.g., SAT-Net). The predicted T₁ maps 204 are used for performance evaluation to compare these different approaches.

Similarly, FIG. 2B compares generating a T₁ map from VFA images and B₁ map using conventional least square fitting, baseline deep learning model, and present model supplementing the VFA images with actual flip angle maps. In FIG. 2B, instead of an uncompensated T₁ map, we predict a compensated T₁ map with B₁ inhomogeneity taken into consideration. Here, the B₁ map is measured using the actual flip angle imaging (AFI) method. In the case of the conventional model, four smoothed VFA diagnostic images 218 are combined with the B₁ map 220 to produce a T₁ map 224 using least squares fitting 222.

In the baseline model, two VFA images 226 are combined with the B₁ map 228 and input to a deep learning network 230 to generate a compensated T₁ map 232. It is significant that this can be performed with only two VFA images as input. In contrast with the baseline method, the present deep learning model 238 predicts the translated images 240 from the same two VFA images 234 supplemented with additional imaging parameter maps 236 that provide the actual flip angles (5° and) 30° at every pixel. The values of nominal flip angles specified by the imaging protocol (i.e., 5° and 30°) are incorporated into network input in the form of actual flip angles, where the actual flip angle is the nominal flip angle modulated by B₁ map, as given by α=αₙₒₘᵢₙₐₗ·B₁. The model derives translated images 240 from two VFA images 234 as well as images 236 that reflect actual flip angles at every pixel. Of note, imaging parameters can be combined with other a priori information (e.g., B₁ map) and used as network input.

FIG. 2C illustrates another example where water and fat images are generated from contrast enhanced dual-echo images. For dual-echo image acquisition, a 3D spoiled-gradient echo sequence is applied with variable density Poisson disc sampling pattern. Based upon prescribed image resolution and system gradient strength, a TE of 2.23 ms for in-phase images and two clusters of TE (1.21-1.31 ms or 3.35 ms) for out-of-phase images are applied for data acquisition at 3T. From k-space data, in-phase and out-of-phase images 242, 250 are reconstructed, and dual-echo complex images are used to produce water and fat images via conventional water-fat separation method (e.g., projected power approach).

Two deep learning-based water-fat separation models are compared in FIG. 2C. Using deep learning, computational time can be significantly reduced, and robustness of water-fat separation can be improved. In the baseline case, in-phase and out-of-phase images 242 are input to the deep neural network 246 to generate fat and water images 248. The baseline network 246 predicts water and fat images from only dual-echo images. In contrast, the present deep neural network 254 derives water and fat images 256 from both dual-echo images 250 and imaging parameter maps 252 that provide the echo times of in-phase and out-of-phase images at every pixel. In our tests, both networks 246, 254 have the same architecture, which is a multi-output T-Net (a densely connected hierarchical convolutional network). Of note, the proposed mechanism can easily work with other network architectures (e.g., attention convolutional network such as SAT-Net, pure attention network such as transformer, or generative adversarial network).

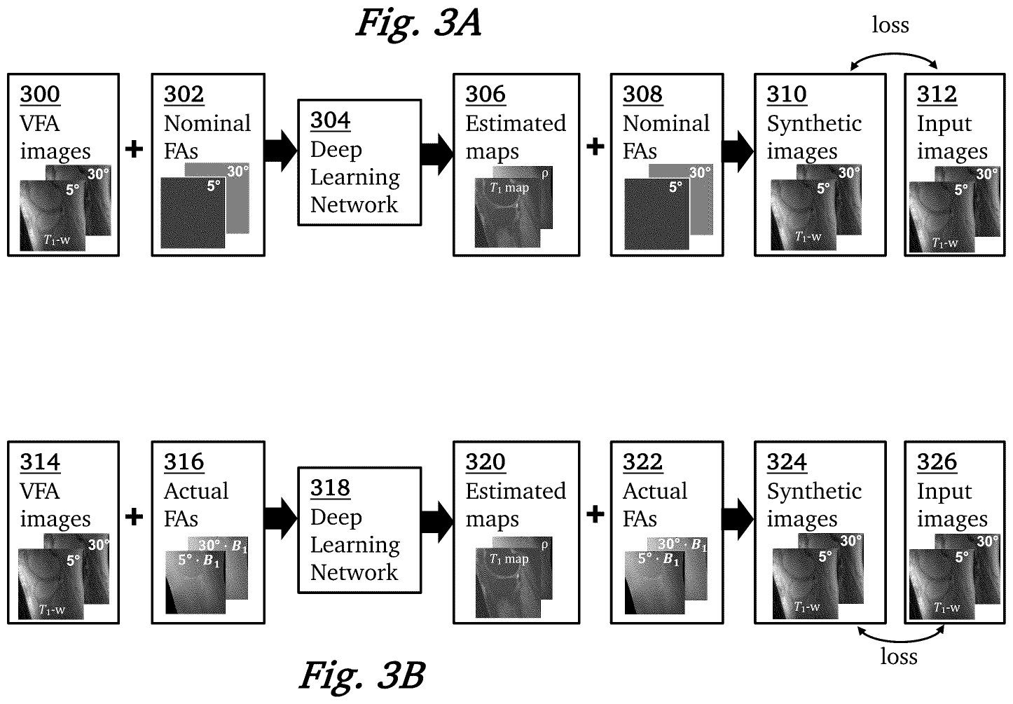

The networks 214, 238, 254 may be trained with diagnostic images and corresponding ground truth translated images (e.g., quantitative parametric maps) that are generated using conventional techniques. For example, the ground truth T₁ maps 204 may be generated from VFA images 200 using least square fitting 202. Preferably, the inventors have developed a self-supervised learning method, which does not require computation of ground truth translated images (e.g., parametric maps). Even when ground truth maps are not used in training, the T₁ maps predicted from two VFA images have high fidelity to the ground truth maps. This training approach is illustrated in FIGS. 3A-3B, which apply the method to uncompensated T₁ mapping and compensated T₁ mapping, respectively. As shown in FIG. 3A, two VFA images 300 and corresponding nominal flip angle imaging parameter maps 302 are applied to the input of deep neural network 304 which outputs estimated uncompensated T₁ and ρ maps 306. These T₁ and ρ maps are then combined with nominal flip angle imaging parameter maps 308 to produce synthetic images 310 which are then compared with the original VFA images 312, and the loss function is back-propagated to train the network 304. Incorporation of nominal flip angles 302 as additional network input provides considerable improvement in prediction. Similarly, as shown in FIG. 3B, two VFA diagnostic images 314 and corresponding actual flip angle maps 316 are applied to the input of deep neural network 318 which outputs estimated compensated T₁ and ρ maps 320. These T₁ and ρ maps are then combined with actual flip angle maps 322 to produce synthetic images 324 which are then compared with the original VFA images 314, and the loss function is back-propagated to train the network 318.

The ρ map, which is used in image synthesis, may be generated together with the T₁ map using a multi-output deep neural network, where different parametric maps are predicted using parallel subnets with distinct encoder and decoder paths. Alternatively, only T₁ map is predicted, and ρ map is calculated from the predicted T₁ map and an input T₁-weighted image (based on the physics model) in every iteration.

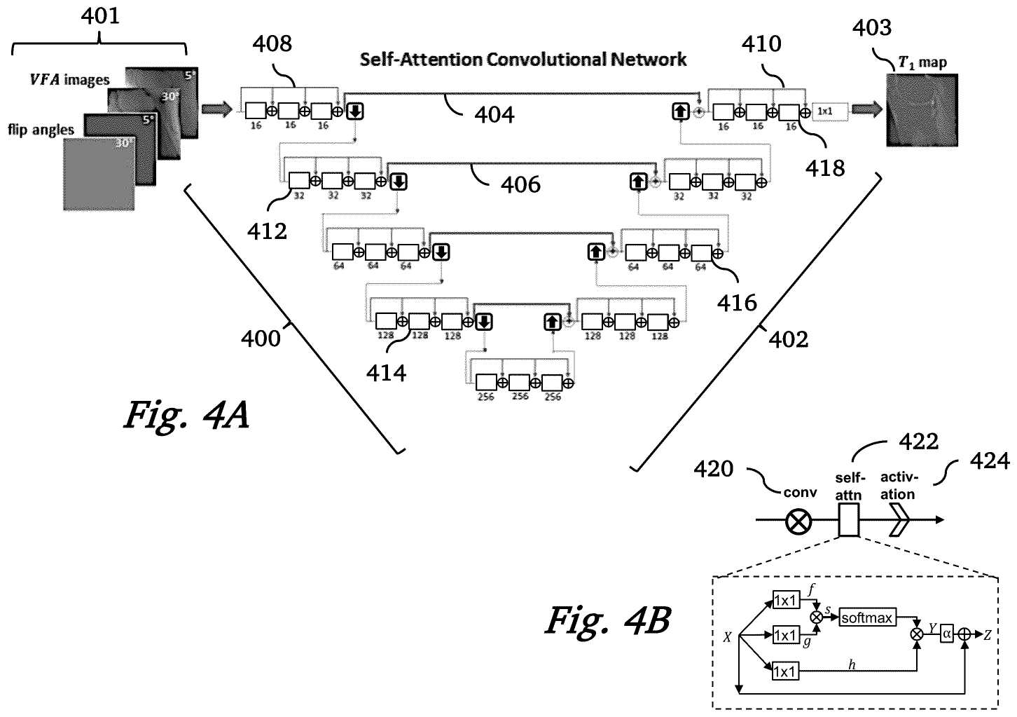

FIG. 4A is a schematic diagram illustrating the architecture of a self-attention network (SAT-net) employed for self-supervised T₁ mapping according to an embodiment of the invention. This hierarchical deep convolutional neural network is composed of an encoder path 400 having four down-samplings and a decoder path 402 having four up-samplings. VFA images and flip angle maps are combined as the input 401 to the top level of the encoder path 400. The network outputs T₁ map 403 from the top level of the decoder path 402. Global shortcuts (e.g., 404, 406) that connect corresponding levels of the encoder path 400 and the decoder path 402 compensate for details lost in down-sampling. Each level of the encoder and decoder has a sequence of three convolutional blocks (e.g., 412, 414, 416, 418). Local shortcuts (e.g., 408, 410) that forward the input to a hierarchical level of a single path to all subsequent convolutional blocks facilitate residual learning. An attention mechanism is incorporated to make efficient use of non-local information.

FIG. 4B is a schematic diagram illustrating the architecture of a single convolutional block in the network of FIG. 4A. Each convolutional block has a convolution layer 420, a self-attention layer 422, and a nonlinear activation layer 424. The self-attention layer 422 has a self-attention map derived by attending to all the positions in the feature map obtained in the previous convolutional layer 420. In this way, direct interactions are established between all voxels within a given image, and more attention is focused on regions that contain similar spatial information. The self-attention mechanism improves the performance of the system by making efficient use of long-range dependencies across image regions.

More specifically, the value at a position of the attention map is determined by two factors. One is the relevance between the signals at current position i and other position j, defined by an embedded Gaussian function

$$s ( {X}_{i} , {X}_{j} ) = exp \{ {( {W}_{f} {X}_{i} )}^{′} ( {W}_{g} {X}_{j} ) \} .$$

The other is a representation of the feature value at the other position j, given by a linear function h(Xⱼ)=WₕXⱼ. Here, W_{f}, W_{g}, and Wₕ are weight matrices (implemented as 1×1 convolution), whose optimal values are identified by the model in training. Within each attention layer, a shortcut connection is established to include local features as well. The contributions of local and non-local information are balanced by a scale parameter a, whose value is obtained in training.

To simultaneously predict T₁ and ρ maps, a multi-output deep neural network may be constructed. The network has parallel subnets with distinct encoder-decoder paths for the generation of individual parametric maps. Each subnet has the network architecture as described in FIGS. 4A-4B.

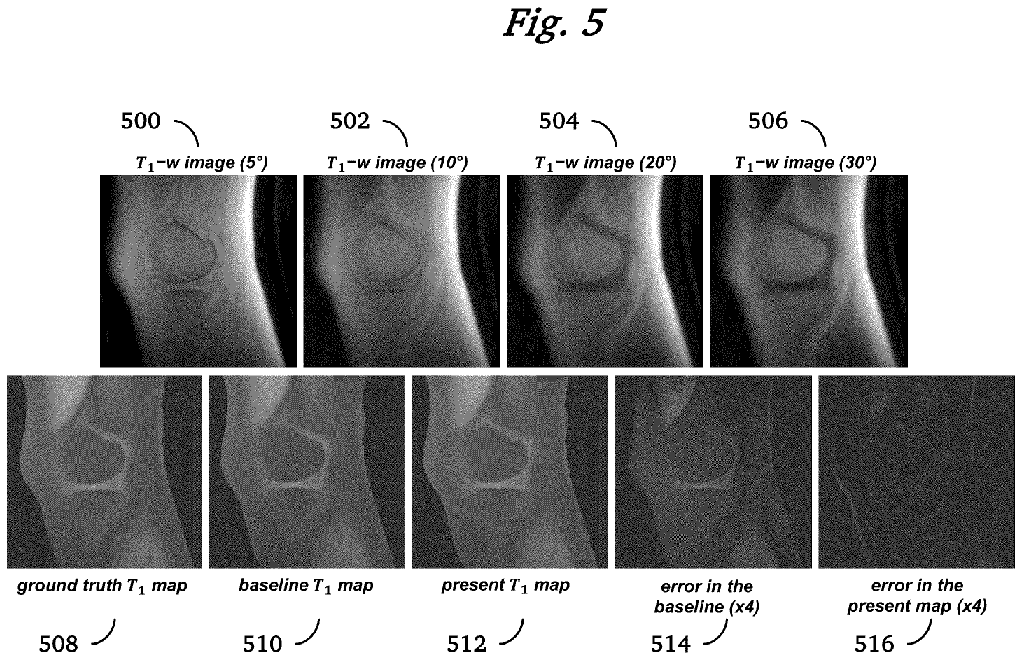

Results demonstrating the performance of embodiments of the present invention are shown in FIGS. 5, 6, 7.

FIG. 5 shows T₁ weighted images 500, 502, 504, 506 acquired with flip angles 5°, 10°, 20°, and 30°, respectively. Also shown are uncompensated T₁ maps 508, 510, 512 corresponding to ground truth, generated using baseline, and generated using the present method, respectively. Images 514, 516, show, respectively, the error between ground truth 508 and baseline 510, and the error between ground truth 508 and the present map 512. These error images 516, 514 demonstrate, respectively, that the uncompensated T₁ map 512 predicted using the present method from two T₁ weighted images and nominal flip angles is more accurate than the T₁ map 510 predicted only from two T₁ weighted images.

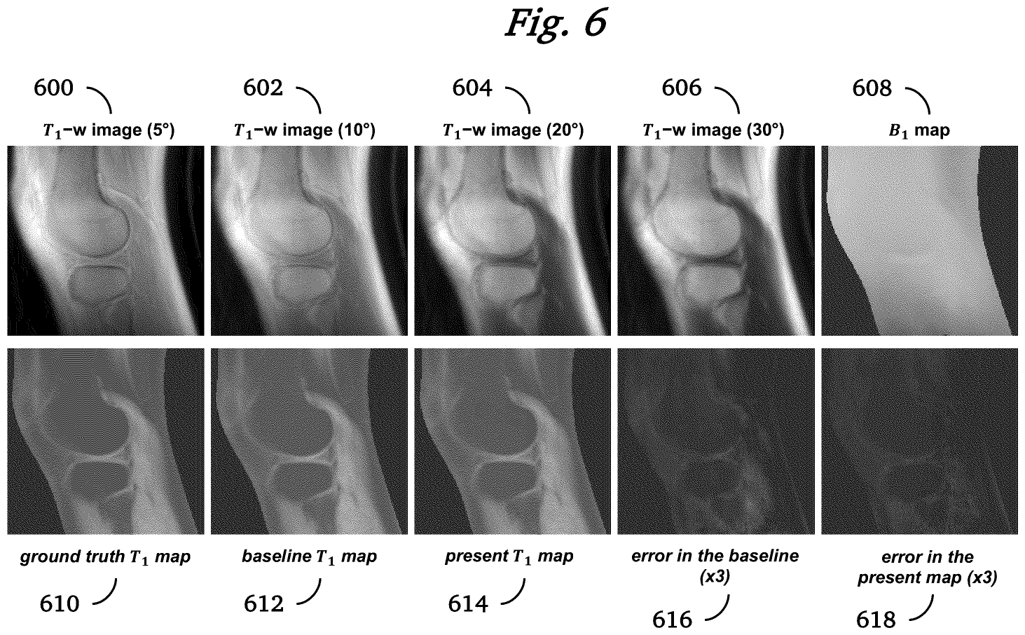

FIG. 6 shows T₁ weighted images 600, 602, 604, 606 acquired with flip angles 5°, 10°, 20°, and 30°, respectively. Also shown is a B₁ map 608 and compensated T₁ maps 610, 612, 614 corresponding to ground truth, generated using baseline, and generated using the present method, respectively. Images 616, 618, show, respectively, the error between ground truth 610 and baseline 612, and the error between ground truth 610 and the present map 614. These error images 618, 616 demonstrate, respectively, that the compensated T₁ map 614 predicted from two T₁ weighted images and actual flip angles is more accurate than the T₁ map 612 predicted from two T₁ weighted images and B₁ map.

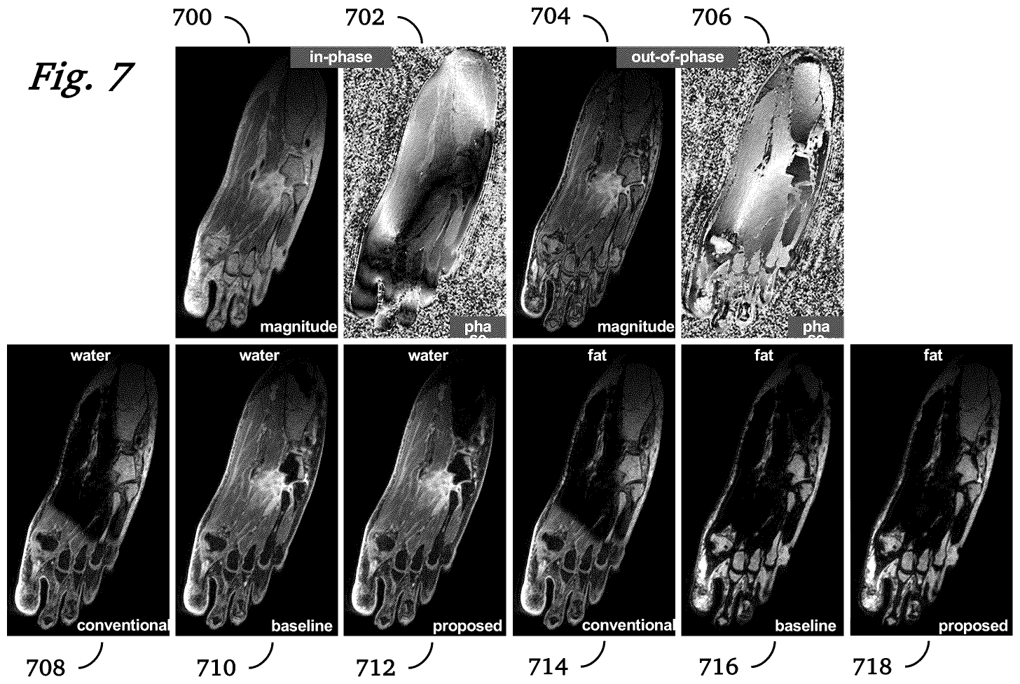

FIG. 7 shows acquired in-phase images 700, 702 and out-of-phase images 704, 706. Also shown are water images 708, 710, 712 predicted using conventional, baseline, and present methods, respectively, as well as fat images 714, 716, 718 predicted using conventional, baseline, and present methods, respectively. These images demonstrate that the water and fat images 712, 718 predicted using the present method from dual echo images and echo times are more accurate than the water and fat images 710, 716 predicted using the baseline method only from dual echo images. Both of them are more accurate than the reference water and fat images 708, 714 obtained using conventional projected power approach.

Significantly, a priori information of the critical imaging parameters is incorporated as additional network input. In fact, this is a new way to make use of a priori information in any deep learning-based medical imaging model. While a medical imaging model can be established without including imaging parameters, explicit provision of such a priori information is expected and has been demonstrated to improve the performance of the system. Imaging parameters can be incorporated in different ways, either contribute as independent images (as in uncompensated T₁ mapping) or combine with other a priori information and form new images (as in compensated T₁ mapping). The mechanism can be applied in supervised or self-supervised learning models, for MR imaging and beyond.

This technique is not limited to the illustrative examples discussed here. Other than VFA-base T₁ mapping, the proposed method can be extended to a variety of quantitative parametric mapping applications, such as Inversion Recovery based T₁ mapping, T₂ or T_{2*} mapping, R₂ or R_{2*} mapping, and T_{1ρ} mapping.

This technique also can be applied in various radiological imaging facilities (e.g., MRI, CT, ultrasound) or image guided therapeutic facilities (e.g., radiation therapy treatment system).

For example, in variable flip angle imaging based T₁ mapping, flip angles can be included; in modified look-locker imaging based T₁ mapping, inversion times can be included; in multi-echo T₂ or T_{2*} mapping, echo times can be included; in continuous wave T_{1ρ} mapping, spin-lock time can be included; in adiabatic Tip mapping, number of adiabatic inversion recovery pulses can be included. In chemical shift encoded water-fat separation, echo times or the difference between the echo times can be included.

This technique can be easily combined with any deep neural network architecture (e.g., convolutional network, attention convolutional network, pure attention network, or generative adversarial network). It can be applied on different radiological imaging modalities. The diagnostic imaging scan may use various different imaging techniques to acquire input images (imaging techniques depend on imaging modality, application, MR pulse sequence, etc.)

The values of various imaging parameters (e.g., flip angles, echo times, inversion times, spin-lock time, number of adiabatic inversion recovery pulses) may be used as network input (the choice of imaging parameters depends on imaging modality, application, MR pulse sequence, etc.). The values of imaging parameters either as individual images or as images that contain other a priori information (e.g., B₁ map) may be used. Deep neural networks with different architectures may be used. Supervised, unsupervised, self-supervised learning models may be used.

1. A method for diagnostic imaging comprising:
performing a diagnostic imaging scan using predetermined image acquisition parameters prescribed in an imaging protocol to produce diagnostic images; and
generating translated diagnostic images from the predetermined image acquisition parameters and from the diagnostic images using a deep neural network;
wherein generating the translated diagnostic images comprises applying both the predetermined image acquisition parameters and the diagnostic images as input to an input layer of the deep neural network;
wherein the predetermined image acquisition parameters are input to the deep neural network in the form of parameter image maps with imaging parameter values at each pixel of the parameter image maps;
wherein the translated diagnostic images are produced as output from an output layer of the deep neural network.

2. The method of claim 1
wherein the diagnostic imaging is magnetic resonance imaging,
wherein the diagnostic images are T₁-weighted images acquired using variable flip angles,
wherein the predetermined image acquisition parameters comprise variable flip angles, and
wherein the translated diagnostic images comprise a T₁ map.

3. The method of claim 2
wherein the diagnostic images are T₁-weighted images acquired with two distinct flip angles,
wherein the translated diagnostic images comprise an uncompensated T₁ map.

4. The method of claim 2
wherein the predetermined image acquisition parameters are combined with a B₁ map to produce actual variable flip angles;
wherein the actual variable flip angles are input into the neural network in the form of a nominal flip angle modulated by the B₁ map;
wherein the translated diagnostic images comprise a compensated T₁ map that takes in account B₁ inhomogeneity.

5. The method of claim 2 wherein the translated diagnostic images comprise a p map.

6. The method of claim 1
wherein the diagnostic imaging is chemical shift encoded magnetic resonance imaging (MRI) using dual echo image acquisition;
wherein the diagnostic images are in-phase and out-of-phase complex MRI images,
wherein the predetermined image acquisition parameters are echo times, and
wherein the translated diagnostic images are water and fat images.

7. The method of claim 1 wherein the diagnostic images are modified look-locker imaging based T₁ weighted images, multi-echo T₂ or T_{2*} weighted images, continuous wave T_{1ρ} weighted images, adiabatic T_{1ρ} weighted images.

8. The method of claim 1 wherein the predetermined image acquisition parameters are inversion times, spin-lock times, echo times, spin-lock times, or number of adiabatic inversion recovery pulses.

9. The method of claim 1 wherein the deep neural network is a convolutional network, attention convolutional network, pure attention network, or generative adversarial network.

10. The method of claim 1 wherein the deep neural network is trained using training diagnostic images and corresponding translated images generated using least square fitting for generating quantitative parametric maps, and projected power approach for generating water and fat images.

11. The method of claim 1 wherein the deep neural network is trained using training diagnostic images via self-supervised learning technique comprising inputting the training diagnostic images to the deep neural network to produce estimated translated images as output, generating from the estimated translated images synthetic images using a model-based calculation, and computing a loss function by comparing the synthetic images to the training diagnostic images.
